(19) United States
(12) Patent Application Publication
(10) Pub. No.: US 2024/0300878 A1
(43) Pub. Date: Sep. 12, 2024

(54) PHOTOCHEMICAL PREPARATION OF FLUORINE-CONTAINING COMPOUNDS

(71) Applicant: WISys Technology Foundation, Inc., Madison, WI (US)

(72) Inventors: Ian Andrew MacKenzie, Platteville, WI (US); Mark Elliot Levenstein, Madison, WI (US)

(21) Appl. No.: 18/444,075

(22) Filed: Feb. 16, 2024

Related U.S. Application Data
(60) Provisional application No. 63/446,129, filed on Feb. 16, 2023.

(51) Int. Cl. C07C 37/18; B01J 31/02; B01J 35/39; C07D 233/64; C07D 249/08; C07D 307/94; C07F 7/12

(52) U.S. Cl. CPC C07C 37/18; B01J 31/0201; B01J 31/0244; B01J 35/39; C07D 233/64; C07D 249/08; C07D 307/94; C07F 7/127; B01J 2231/4288

(57) ABSTRACT

Various embodiments disclosed relate to a method of preparing aryl fluorinated ether compounds. The method involves contacting an aryl halide with a fluorinated alcohol in the presence of a photocatalyst, a base, and irradiation with electromagnetic radiation comprising a wavelength between about 200 nm and about 800 nm. The present invention also provides a method of late-stage photochemical modification of a biologically active compound, such as drugs or agrochemicals. Fluorinated derivatives of griseofulvin, clofibrate, and 2,4-D methyl ester are described herein.

### CROSS-REFERENCE TO RELATED APPLICATION

This application claims the benefit of priority to U.S. Provisional Patent Application Ser. No. 63/446,129 entitled “PHOTOCHEMICAL PREPARATION OF FLUORINE-CONTAINING COMPOUNDS,” filed Feb. 16, 2023, the disclosure of which is incorporated herein in its entirety by reference.

### BACKGROUND

The element fluorine has acquired an ever-increasing prevalence in synthetic drugs and agrochemicals due to its impact on metabolism, chemical stability, and modulating lipophilicity. It is estimated that 20 to 30 percent of marketable drugs contain at least one fluorine atom. Some fluorine-containing chemicals demonstrate greater biological activity and resistance to metabolism. In agriculture, fluorine-containing functional groups are appealing in potential herbicides, pesticides, and fungicides. Over the past two decades, more than half of newly registered agrochemical actives have contained at least one fluorine atom.

However, there are challenges to introducing fluorine into pharmaceutical and agrochemical structures, particularly when there is a desire to do so in the later steps of chemical synthesis or via modification of an existing active ingredient. Fluorination can require harsh reaction conditions, high temperatures, expensive reagents, toxic reagents, or transition metal catalysts. There is a need to improve the functionality of existing drugs and agrochemicals and to develop new techniques for introducing fluorinated functional groups into molecules.

### SUMMARY OF THE INVENTION

The present disclosure provides a method for preparing a fluorinated compound, comprising contacting an aryl halide with a fluorinated alcohol in the presence of a photocatalyst, a base, and irradiation with electromagnetic radiation comprising a wavelength between about 200 nm and about 800 nm.

The present disclosure also provides a method of late-stage photochemical modification of a biologically active compound, comprising: selecting a biologically active compound having an aryl halide; and contacting the biologically active compound with a fluorinated alcohol in the presence of a photocatalyst, a base, and irradiation with electromagnetic radiation comprising a wavelength between about 200 nm and about 800 nm.

The present disclosure also provides a fluorinated derivative of griseofulvin having the structure:

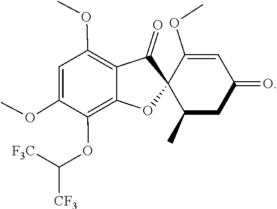

Existing approaches to incorporating fluorinated groups onto aromatic rings can involve harsh conditions, toxic reagents, or transition metals. Such aspects can render existing methods unsuitable for late-stage modification of biologically active compounds. To address these problems, the present disclosure provides a light-mediated nucleophilic aromatic substitution reaction of aryl halides. The presently disclosed reaction permits access to new fluorinated derivatives of agrochemical and pharmaceutical actives.

The presently described method can involve inexpensive starting materials, an organic photocatalyst, visible light, room temperature (or slightly above due to moderate heat from the lights) and simple sodium bicarbonate as a base. Hexafluoroisopropanol (HFIP) is used as a representative nucleophile, which provides access to desirable aryl fluoroalkyl ethers. The photocatalyst can be metal-free and used in substoichiometric amounts. Numerous aryl halide substrates are available, including biologically active aryl halides, thus permitting access to large numbers of new compounds having potentially desirable properties. For example, the presently described method can be used for direct functionalization of a variety of existing pesticides and antifungals. This methodology thus represents a platform for late-stage modification of drugs, agrochemicals, or synthetic intermediates to include fluorine and other important functional moieties.

### BRIEF DESCRIPTION OF FIGURES

FIG. 1 provides a chemical structure illustrating a pyrylium-type photocatalyst and three charts illustrating how electronic properties (UV-Vis Absorption Spectra, Emission Spectra, and Cyclic Voltammetry) differ as R groups are changed on the photocatalyst.

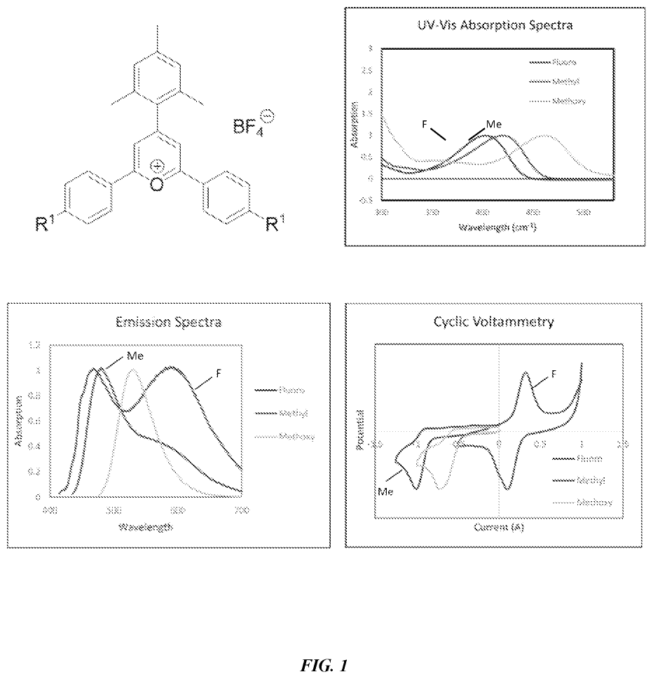

FIG. 2 provides an illustration showing a proposed catalytic cycle for the observed transformations.

### DETAILED DESCRIPTION OF THE INVENTION

The present disclosure provides methods useful for obtaining fluorine-containing compounds. For example, the presently disclosed method permits access to fluorine-containing derivatives of biologically active compounds.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

### Definitions

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. For example, an oxygen-containing group such as an alkoxy group, aryloxy group, arylalkyloxy group, oxo(carbonyl) group, a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring (carboxyclic aromatic rings) and cyclic aromatic groups that contain one or more heteroatom in the ring (heteroaromatic rings). Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “arylhalide” refers to an aryl substituted with one or more chloride, bromide, iodide, fluoride, or a halogen-equivalent of the same (e.g., Triflate). The term “aryl chloride” refers to an aryl substituted with one or more chloride. For example, a chlorobenzene, which may be further substituted by any number of additional substituents.

The term “heterocyclyl” as used herein refers to aromatic and non-aromatic ring compounds containing three or more ring members, of which one or more is a heteroatom such as, but not limited to, N, O, and S.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “hydrocarbon” or “hydrocarbyl” as used herein refers to a molecule or functional group, respectively, that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids. Examples of organic solvents are THF, diethyl ether, benzene, toluene, dichloromethane, hexane, acetone, acetonitrile, ethanol, formaldehyde, chloroform, carbon tetrachloride, and acetic acid. The present conducted reactions may be conducted in a solvent. In other embodiments, the reaction may be conducted neat.

The term “room temperature” as used herein refers to a temperature of about 15° C. to 28° C.

The term “standard temperature and pressure” as used herein refers to about 20° C. and about 101 kPa.

Ambient temperature refers to a temperature around room temperature, corresponding to the indoor or outdoor environment in which the process is conducted, and in which its vessel is not subjected to additional external cooling or heating efforts. Ambient pressure refers to a pressure around 101 kPa (around sea-level) to about 90 kPa (at about a mile above sea level), corresponding to the pressure of the indoor or outdoor environment, and in which the vessel is not subjected to additional external pressurizing or depressurizing efforts. For example, ambient pressure can be p=101325×(1−2.25577×10⁻⁵×h)^{5.25588} in which h is altitude above sea level in meters and p is pressure (Pa). Ambient air can have a molecular oxygen content of about 10 to about 30 vol %, about 15 to about 25 vol %, about 18 to about 23 vol %, about 19 vol % to about 22 vol %, about 20 vol % to about 22 vol %, about 20.5 vol % to 21.5 vol %, about 20 vol %, about 21 vol %, or about 22 vol %. In various embodiments, ambient air can have a molecular oxygen content of about 20.95% by volume.

In various embodiments, salts having a positively charged counterion can include any suitable positively charged counterion. For example, the counterion can be ammonium (NH₄⁺), or an alkali metal such as sodium (Na⁺), potassium (K⁺), or lithium (Li⁺). In some embodiments, the counterion can have a positive charge greater than +1, which can in some embodiments complex to multiple ionized groups, such as Zn²⁺, Al³⁺, or alkaline earth metals such as Ca²⁺ or Mg²⁺.

In various aspects, salts having a negatively charged counterion can include any suitable negatively charged counterion. For example, the counterion can be boron tetrafluoride (BF₄⁻), perchlorate (ClO₄⁻), sulfate, carbonate, bicarbonate, chloride, bromide, iodide, nitrate, and triflate.

### Photochemical Preparation of Fluorinated Alkyl Aryl Ethers

The present disclosure provides a photochemical process utilizing mild reaction conditions for obtaining fluorine-containing compounds.

In various aspects, the process utilizes an aryl halide as the starting material substrate. The aryl halide can comprise a number of substituents and the aryl ring may be fused to one or more other rings. The aryl can be a benzene ring, a naphthalene ring, or a heteroaromatic ring. The aryl halide can be substituted with one, two, three or more halides.

The aryl halide can further comprise one or more additional substituents. The one or more additional substituents can be an activating group, a deactivating group, a group other than an activating group, or a group other than a deactivating group. Examples activating groups include alkoxide, hydroxy, alkoxy, amine, amide, ester, or alkyl. Examples of deactivating groups include fluorine, bromine, iodo, carbonyl, sulfonyl, cyano, nitro, haloalkyl, or ammonium. The one or more additional substituents can be an electron-donating group, an electron-withdrawing group, a group other than an electron-donating group, or a group other than an electron-withdrawing group. In various further aspects, the one or more additional substituents can be alkoxy. In some aspects, the one or more additional substituents can be alkyl, aryl, or other hydrocarbon groups. The one or more additional substituents can be methyl.

The aryl halide can be an aryl chloride.

The aryl halide can be a bioactive compound, such as a pharmaceutical or agrochemical compound. Examples of bioactive aryl chlorides include griseofulvin, diclofenac, triclosan, 2,4,5-Trichlorophenoxyacetic acid, vancomycin, DDT, dicofol, pentachlorphenol, vinclozolin, tetradifon, chlorpyrifos, atrazine, triadimefon, climbazole, derivatives thereof, and the like.

In various aspects, the process utilizes a fluorinated alcohol as the source of the fluorinated group. The resulting fluorinated product is an aryl fluorinated alkyl ether.

The fluorinated alcohol can be a fluorinated C1-C8 alcohol. The fluorinated alcohol can have 1 to 15 fluorines. The alcohols can be primary, secondary, or tertiary alcohols. In various aspects, the fluorinated alcohol is a secondary alcohol. As such, it can have the structure HO—CH—((CR₂)ₙ—CR₃)₂, wherein R is H or F, and n is 0 to 3. In another aspect, the fluorinated alcohol can be a primary alcohol; as such, it can have the structure HO—(CR₂)ₙ—CR₃, wherein R is H or F, and n is 0 to 7. In yet another aspect, the fluorinated alcohol can be a tertiary alcohol; as such, it can have the structure HO—C—((CR₂)ₙ—CR₃)₃, wherein R is H or F, and n is 0 to 7. In various aspects, the fluorinated alcohol is fully fluorinated at each carbon, or fully fluorinated at each carbon except for the carbon to which the alcohol is attached. In various aspects, the fluorinated alcohol is fully fluorinated except that it comprises one CH bond. In various aspects, the fluorinated alcohol can have the structure:

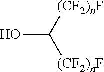

wherein n is 1 to 3.

The fluorinated alcohol can be hexafluoroisopropanol.

In yet further aspects, non-alcohol fluorinated nucleophiles can be used.

In various aspects, the process utilizes an organic photocatalyst. The photocatalyst can be free of transition metals. In some aspects, the photocatalyst can be free of toxic metals. In yet further aspects, the photocatalyst can be free of any metals. The photocatalyst can comprise an aromatic cation in conjugation with one or more aromatic rings or other extended pi-systems.

For example, the photocatalyst can be a pyrylium salt. The pyrylium salt can have the structure:

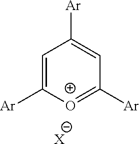

wherein Ar is an aryl group and X is an anionic counterion.

As another example, the pyrylium salt can have the structure:

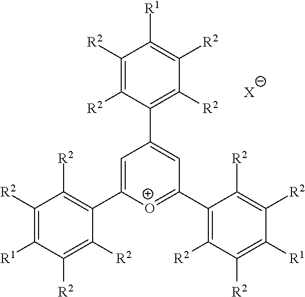

wherein
R₁ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl;
R₂ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl; and
X is an anionic counterion.

As another example, the pyrylium salt can have the structure:

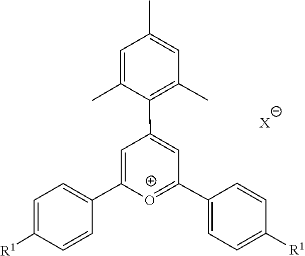

wherein R₁ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl, and X is an anionic counterion.

The photocatalyst can also be an acridinium salt. The acridinium salt can have the structure:

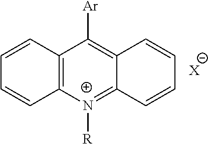

wherein Ar is an aryl group, R is alkyl or aryl, and X is an anionic counterion.

For example, the acridinium salt can have the structure:

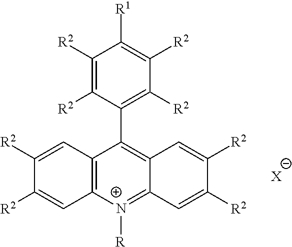

wherein R₁ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl, R₂ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl; and R is alkyl or aryl, and X is an anionic counterion.

As yet another example, the acridinium salt can have the structure:

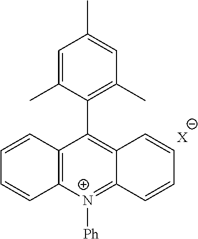

wherein X is an anionic counterion.

Examples of anionic counterions X⁻ can include BF₄⁻, ClO₄⁻, Cl⁻, Br⁻, F⁻, and HCO₃⁻.

The photocatalyst can be used in a sub-stoichiometric, i.e., catalytic, amount. For example, the photocatalyst can be used in amount less than, at least, or approximately 0.01 mol %, 0.02 mol %, 0.03 mol %, 0.04 mol %, 0.05 mol %, 0.06 mol %, 0.07 mol %, 0.08 mol %, 0.09 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, or 50 mol % relative to the aryl halide. For example, the photocatalyst can be used in an amount less than 10 mol % relative to the aryl halide. As another example, the photocatalyst can be used at about 1 mol %, 2 mol %, 3 mol %, 4 mol %, 5 mol %, or 6 mol % relative to the aryl halide. In various aspects that amount of photocatalyst used is less than 1 mg.

In various aspects, the process utilizes a mild base to catalyze the reaction. The base can be an organic base or an inorganic base. For example, the organic base can be a non-nucleophilic amine base. For example, the base can be a tertiary amine such as N,N-diisopropylethylamine. In yet further aspects, the base can be a mildly basic inorganic salt. The base can be an alkali carbonate, alkali earth carbonate, or an alkali bicarbonate. For example, the base can be sodium bicarbonate (NaHCO₃, baking soda).

The base can be used in a stoichiometric quantity, for example, as one or more equivalents relative to the aryl halide. For example, the base can be used as 1, 2, 3, 4, 5, 6, 7, 8, 9, or 10 equivalents relative to the aryl halide. As another example, the base can be used at 2 equivalents relative to the aryl halide. In yet other aspects, the base can be used in a sub-stoichiometric quantity. For example, the base can be used at a 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1 ratio relative to the aryl halide.

In various aspects, the base can provide mild conditions and can have a near neutral pH. For example, the base can have a pH (as determined in a 1% aqueous solution at 25° C.) of at least, less than, or approximately 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11.0. For example, the pH can be around 8 to around 8.5.

In various aspects, the process involves irradiating a reaction mixture comprising the aryl halide, fluorinated alcohol, base, and photocatalyst. The irradiation can be provided in a photoreactor, lamps, LEDs, or a combination thereof. The irradiation can provide light having a wavelength between about 200 nm to about 800 nm. The light can have a wavelength of peak intensity at approximately 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm, 580 nm, 585 nm, 590 nm, 595 nm, 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm, 685 nm, 690 nm, 695 nm, 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm, 785 nm, 790 nm, 795 nm, or 800 nm. In various aspects the light has a peek intensity of between 300 nm and 700 nm, or 400 nm and 500 nm.

In further examples, the irradiation provides light at wavelength of at least, less than, or approximately 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, 430 nm, 431 nm, 432 nm, 433 nm, 434 nm, 435 nm, 436 nm, 437 nm, 438 nm, 439 nm, 440 nm, 441 nm, 442 nm, 443 nm, 444 nm, 445 nm, 446 nm, 447 nm, 448 nm, 449 nm, 450 nm, 451 nm, 452 nm, 453 nm, 454 nm, 455 nm, 456 nm, 457 nm, 458 nm, 459 nm, or 460 nm. The light can be broad spectrum visible light or narrow spectrum light focused on one or more regions of peak intensity.

In various aspects, the process involves contacting the aryl halide with the fluorinated alcohol in the presence of a base and a photocatalyst. The contacting step can be conducted at mild temperatures. For example, the contacting step can be conducted at a temperature around room temperature, or for example 25° C. In further examples, the contacting step can be conducted at temperature of about 40° C. or less, about 35° C. or less, about 25° C. or less. In yet further examples, the contacting step is conducted at a temperature of about 0° C. or greater, about 10° C. or greater, or about 20° C. or greater.

In various aspects, the process involves irradiating the aryl halide and/or fluorinated alcohol in the presence of the photocatalyst and the base. The irradiating step can be conducted at mild temperatures. For example, the irradiating step can be conducted at a temperature around room temperature, or for example 25° C. In further examples, the irradiating step can be conducted at temperature of about 40° C. or less, about 35° C. or less, about 25° C. or less. In yet further examples, the irradiating step is conducted at a temperature of about 0° C. or greater, about 10° C. or greater, or about 20° C. or greater. In various aspects, irradiating the reaction mixture heats up the reaction mixture and external cooling or fans are used to cool the mixture.

In various aspects, the aryl halide and fluorinated alcohol are irradiated while in contact together in the presence of the photocatalyst.

In various aspects, the methods of this disclosure are performed in the absence of any transition metal.

In various aspects, the methods of this disclosure are performed in the absence of any additional catalyst other than the photocatalyst or source of fluorinated component other than the fluorinated alcohol.

The disclosure also provides a method of late-stage photochemical modification of a biologically active compound, comprising:
selecting a biologically active compound having an aryl chloride or other aryl halide;
contacting the biologically active compound with a fluorinated alcohol in the presence of a photocatalyst, a base, and irradiation with electromagnetic radiation comprising a wavelength between about 200 nm and about 800 nm, so as to obtain a fluorinated derivative of the biologically active compound.

In various aspects, the biologically active compound is a pharmaceutical, an agrochemical, or a cleaning product. For example, the biologically active compound can be a bioactive aryl halide, such as griseofulvin, diclofenac, triclosan, 2,4,5-Trichlorophenoxyacetic acid, vancomycin, DDT, dicofol, dicamba, MCPA, and triclopyrpentachlorphenol, vinclozolin, tetradifon, chlorpyrifos, atrazine, triadimefon, clofibrate, climbazole, 2,4-D, derivatives thereof, and the like. This disclosure contemplates that such derivatives would retain the biological activity of the original compound.

In various aspects, the biologically active compound is griseofulvin.

The disclosure also provides a fluorinated derivative of griseofulvin having the structure:

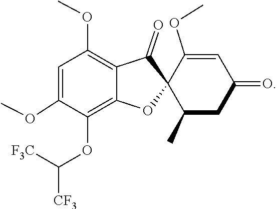

In various aspects, the biologically active compound is clofibrate.

The disclosure also provides a fluorinated derivative of clofibrate having the structure:

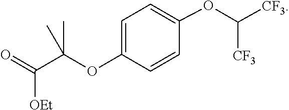 [SMILES: CCOC(=O)C(C)(C)Oc1ccc(OC(C(F)(F)F)C(F)(F)F)cc1]

In various aspects, the biologically active compound is 2,4-D methyl ester.

The disclosure also provides a fluorinated derivative of 2,4-D methyl ester having the structure:

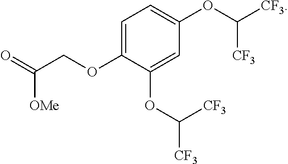 [SMILES: COC(=O)COc1ccc(OC(C(F)(F)F)C(F)(F)F)cc1OC(C(F)(F)F)C(F)(F)F]

### EXAMPLES

Various embodiments of the present invention can be better understood by reference to the following Examples which are offered by way of illustration. The present invention is not limited to the Examples given herein.

### Materials

### Photocatalysts

The experimental examples utilized a variety of organic photocatalysts to investigate and, ultimately, demonstrate the versatility of the presently described method. The choice of suitable photocatalyst is not intended to be particularly limited. Various salts forms can be used. Some examples of photocatalysts offered a maximum absorbance at around 420 nm-440 nm, which is the wavelength of light provided by the photoreactor. The examples herein describe pyrylium and acridinium compounds as representative photocatalysts.

### Pyryliums

The tested pyryliums were generated by the following synthetic approach:

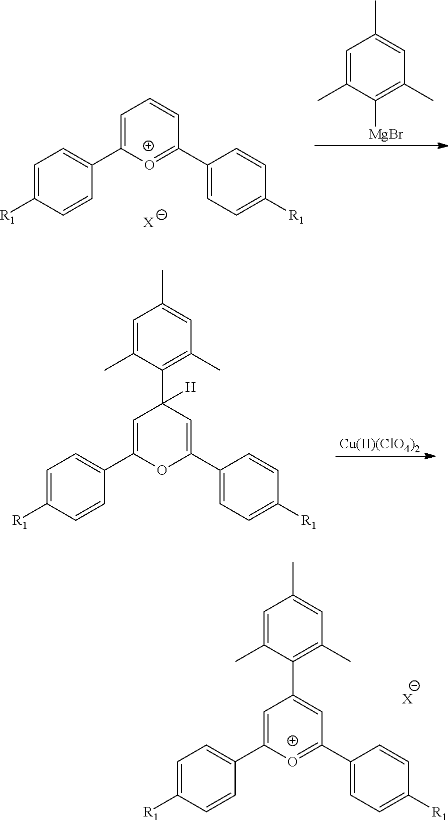

Pyrylium compounds can exist in salt form and neutral form. When in neutral form, the central oxygen-containing heterocycle is a non-aromatic pyran moiety. When in salt form, the central oxygen-containing heterocycle is an aromatic pyrylium moiety. Pyrylium salts can include any number of negative counterions, X⁻. Examples include tetrafluoroborate, halide, perchlorate, and the like.

### Acridiniums

The tested acridiniums were commercially available from Sigma-Aldrich (St. Louis, MO).

### Photocatalyst Selection

A library of modular photocatalysts was developed that would be simple to modify and to activate under different wavelengths. Three examples include methyl-, fluoro-, and methoxy-substituted pyryliums. These photocatalysts were characterized in terms of electrochemistry, fluorescence, UV-Vis and lifetime analysis. These catalysts represent a low-cost catalyst, due to their inexpensive, readily obtained starting materials and minimal steps, and due to being free from transition metal components.

### Photocatalyst Characterization

Modifying the para-substituent of aryl groups at position 2 and 6 of the pyrylium altered electronic properties and thus permitted modulation of electrochemistry, fluorescence, and UV-Vis properties. Results are provided in Table 1, below.

TABLE 1

| Substituent | ABS(max) | EMS(max) | E(0.0 | E(red) | E*(red) | Lifetime |
|---|---|---|---|---|---|---|
| Fluoro | 403 nm | 486 nm | 2.85 V | 0.21 V | 3.06 V | 3 ns |
| Methyl | 421 nm | 479 nm | 2.75 V | −0.90 V | 1.86 V | 3 ns |
| Methoxy | 461 nm | 464 nm | 2.49 V | −0.55 V | 1.94 V | 3 ns |

### Formation of Aryl Fluorinated Ethers

### General Synthetic Method

All reactions were completed by combining starting materials into a vessel, which is placed in a photoreactor, and irradiating the vessel through its bottom with 440 nm light for 12 hours. To assist the LEDs in light permeation, the lateral surface of the vial was coated with tin foil. Overheating was mitigated with the use of fans adjacent to and on top of the photoreactor. Exemplary reactions were performed on a 0.3 mmol scale.

### Development of Reaction Conditions

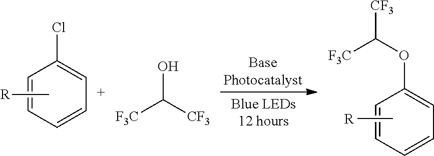

Optimization experiments were set up to explore yield optimizations for the mild photocatalytic aryl fluorinated ether formation. As starting materials, 1-chloro-4-methoxybenzene and 1,1,1,3,3,3-hexafluoropropan-2-ol were utilized as model components. Reaction conditions involved use of a photocatalyst, base, and irradiation with 440 nm light for 12 hours and resulted in the desired 1-((1,1,1,3,3,3-hexafluoropropan-2-yl)oxy)-4-methoxybenzene at 62% yield. Argon gas purging was utilized to provide an inert atmosphere. Irradiation was provided through the bottom of the reaction vessel utilizing 440 nm light from blue LEDs in a photoreactor. To assist with light permeation, the sides of the vessel were provided a reflective foil coating. Fans were used to mitigate heating. Unless otherwise indicated, HFIP was used at 0.3M.

Photocatalysts were utilized in substoichiometric amounts; 1 mol % to 4 mol %. Four catalyst types were tested, shown in Table 2, below.

TABLE 2

| | |
|---|---|
| 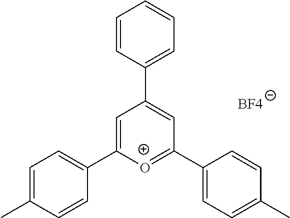 | Catalyst A |
| 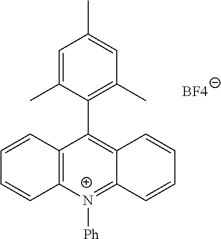 [SMILES: Cc1cc(C)c(-c2c3ccccc3[n+](-c3ccccc3)c3ccccc23)c(C)c1 ; "BF4"] | Catalyst B |
| 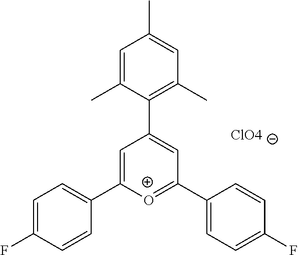 | Catalyst C |
| 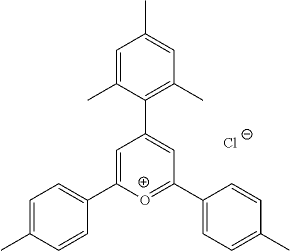 | Catalyst D |

Both organic and inorganic bases were tested. To represent an organic base, DIPEA (N,N-diethylisopropylamine) was used. To represent inorganic bases, K₂CO₃ and NaHCO₃ were used. The base was provided at an amount between equivalents.

Results are provided in Table 3, below.

TABLE 3

| Entry | Photocatalyst | Base | % Yield |
|---|---|---|---|
| 1* | A (2 mol %) | DIPEA (3 eq) | 0 |
| 2* | B (2 mol %) | DIPEA (3 eq) | 19 |
| 3 | B (2 mol %) | DIPEA (3 eq) | 50 |
| 4 | B (2 mol %) | K₂CO₃ (3 eq) | 40 |
| 5 | B (4 mol %) | NaHCO₃ (3 eq) | 48 |
| 6 | B (2 mol %) | NaHCO₃ (3 eq) | 52 |
| 7 | B (1 mol %) | NaHCO₃ (3 eq) | 62 |
| 8 | B (1 mol %) | NaHCO₃ (2 eq) | 62 |
| 9 | B (1 mol %) | K₂CO₃ (2 eq) | 54 |
| 10 | B (1 mol %) | Na₂CO₃ (2 eq) | 53 |
| 11 | A(1 mol %) | NaHCO₃ (2 eq) | 27 |
| 12 | C (1 mol %) | NaHCO₃ (2 eq) | 30 |
| 13 | D (1 mol %) | NaHCO₃ (2 eq) | 20 |

*= entries with a star correspond to reaction conditions in air, whereas the other reactions correspond to an inert environment due to purging the vessel with argon prior to placement in the photoreactor.

Use of an acridinium photocatalyst (Catalyst B, 1 mol %, less than 1 mg) with NaHCO₃ (2 eq.) and irradiation with 440 nm light for 12 hours resulted in the desired 1-((1,1,1,3,3,3-hexafluoropropan-2-yl)oxy)-4-methoxybenzene at 62% yield. Compared to other conditions resulted in improved yield (19% to 62% yield). This improvement can be credited to both starting material selections and also the use of argon gas.

### Developing Substrate Breadth

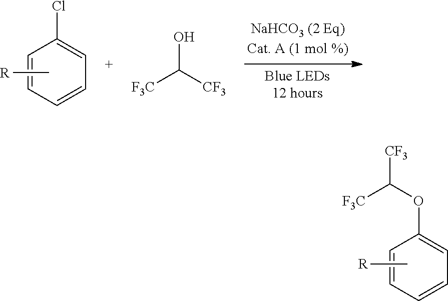

Additional experiments were conducted to develop the breadth of scope of the photocatalytic aryl fluorinated ether formation.

TABLE 4

| Example Starting Material | Product (Yield %) |
|---|---|
| 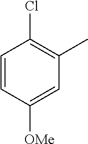 | 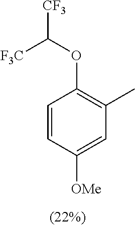 [SMILES: COc1ccc(OC(C(F)(F)F)C(F)(F)F)c(C)c1] |
| 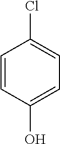 | 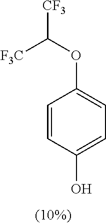 [SMILES: Oc1ccc(OC(C(F)(F)F)C(F)(F)F)cc1] |
| 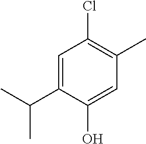 | 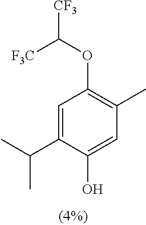 [SMILES: Cc1cc(O)c(C(C)C)cc1OC(C(F)(F)F)C(F)(F)F] |
| 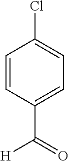 | 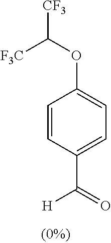 [SMILES: O=Cc1ccc(OC(C(F)(F)F)C(F)(F)F)cc1] |

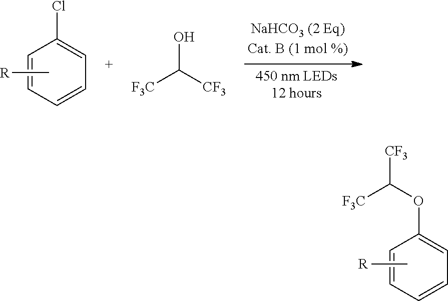

TABLE 5

| | |
|---|---|
| Example Starting Material | Product (Yield %) |
| 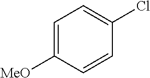 | 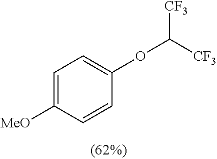 [SMILES: COc1ccc(OC(C(F)(F)F)C(F)(F)F)cc1] |
| 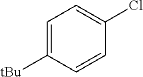 [SMILES: CC(C)(C)c1ccc(Cl)cc1] | 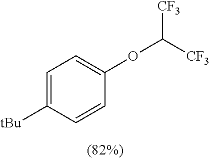 [SMILES: CC(C)(C)c1ccc(OC(C(F)(F)F)C(F)(F)F)cc1] |
| 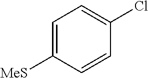 | 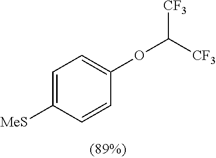 [SMILES: CSc1ccc(OC(C(F)(F)F)C(F)(F)F)cc1] |
| 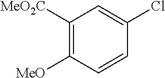 [SMILES: COC(=O)c1cc(Cl)ccc1OC] | 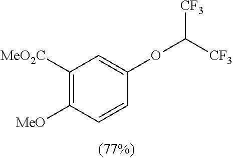 [SMILES: COC(=O)c1cc(OC(C(F)(F)F)C(F)(F)F)ccc1OC] |
| 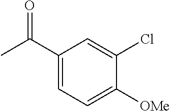 | 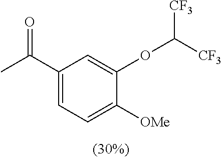 [SMILES: COc1ccc(C(C)=O)cc1OC(C(F)(F)F)C(F)(F)F] |
| 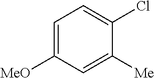 | 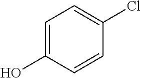 |
| 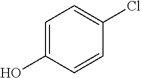 | 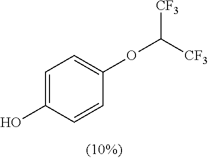 [SMILES: Oc1ccc(OC(C(F)(F)F)C(F)(F)F)cc1] |
| 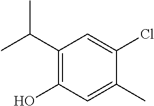 | 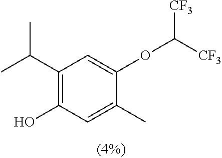 [SMILES: Cc1cc(O)c(C(C)C)cc1OC(C(F)(F)F)C(F)(F)F] |
| 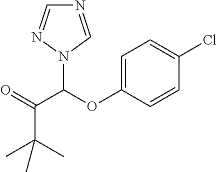 [SMILES: CC(C)(C)C(=O)C(Oc1ccc(Cl)cc1)n1cncn1] | 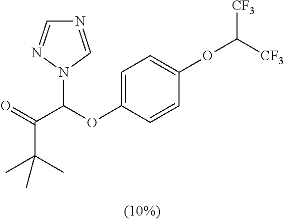 [SMILES: CC(C)(C)C(=O)C(Oc1ccc(OC(C(F)(F)F)C(F)(F)F)cc1)n1cncn1] |
| 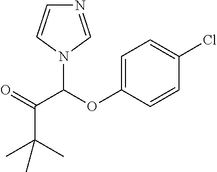 [SMILES: CC(C)(C)C(=O)C(Oc1ccc(Cl)cc1)n1ccnc1] | 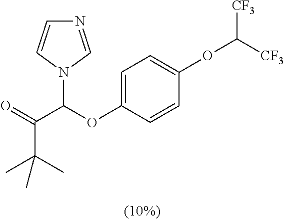 [SMILES: CC(C)(C)C(=O)C(Oc1ccc(OC(C(F)(F)F)C(F)(F)F)cc1)n1ccnc1] |
| 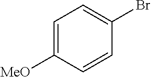 | 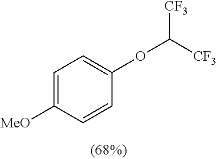 [SMILES: COc1ccc(OC(C(F)(F)F)C(F)(F)F)cc1] |
| 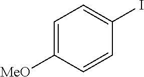 | 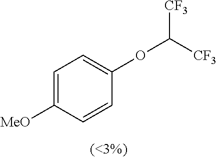 [SMILES: COc1ccc(OC(C(F)(F)F)C(F)(F)F)cc1] |
| 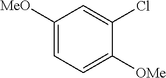 [SMILES: COc1ccc(OC)c(Cl)c1] | 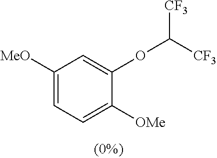 [SMILES: COc1ccc(OC)c(OC(C(F)(F)F)C(F)(F)F)c1] |
| 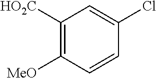 [SMILES: COc1ccc(Cl)cc1C(=O)O] | 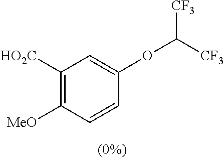 [SMILES: COc1ccc(OC(C(F)(F)F)C(F)(F)F)cc1C(=O)O] |
| Product (Yield %) | Product (Yield %) |
| 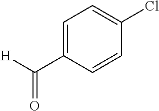 | 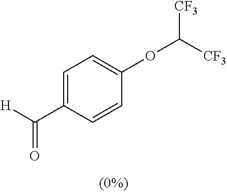 [SMILES: O=Cc1ccc(OC(C(F)(F)F)C(F)(F)F)cc1] |
| 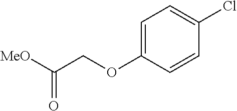 [SMILES: COC(=O)COc1ccc(Cl)cc1] | 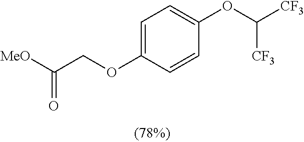 [SMILES: COC(=O)COc1ccc(OC(C(F)(F)F)C(F)(F)F)cc1] |
| 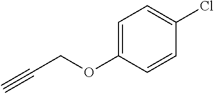 | 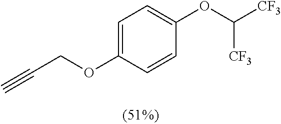 [SMILES: C#CCOc1ccc(OC(C(F)(F)F)C(F)(F)F)cc1] |
| 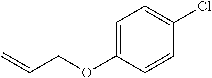 | 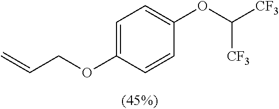 [SMILES: C=CCOc1ccc(OC(C(F)(F)F)C(F)(F)F)cc1] |
| 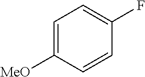 | 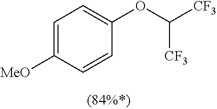 [SMILES: COc1ccc(OC(C(F)(F)F)C(F)(F)F)cc1] |
| 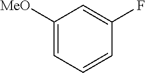 | 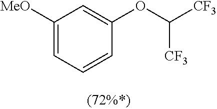 [SMILES: COc1cccc(OC(C(F)(F)F)C(F)(F)F)c1] |
| 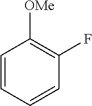 | 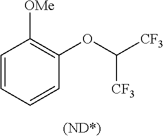 [SMILES: COc1ccccc1OC(C(F)(F)F)C(F)(F)F] |
| 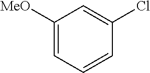 | 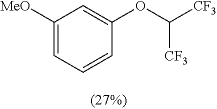 [SMILES: COc1cccc(OC(C(F)(F)F)C(F)(F)F)c1] |
| 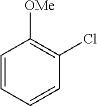 | 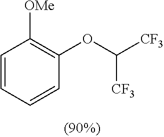 [SMILES: COc1ccccc1OC(C(F)(F)F)C(F)(F)F] |
| 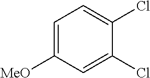 | 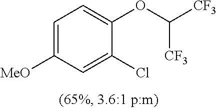 [SMILES: COc1ccc(OC(C(F)(F)F)C(F)(F)F)c(Cl)c1] |
| 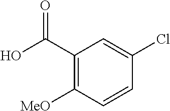 [SMILES: COc1ccc(Cl)cc1C(=O)O] | 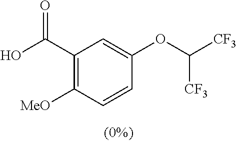 [SMILES: COc1ccc(OC(C(F)(F)F)C(F)(F)F)cc1C(=O)O] |
| 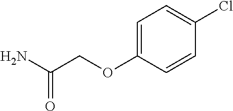 [SMILES: NC(=O)COc1ccc(Cl)cc1] | 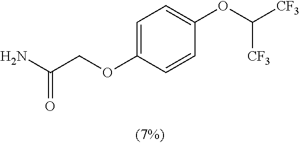 [SMILES: NC(=O)COc1ccc(OC(C(F)(F)F)C(F)(F)F)cc1] |
| 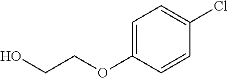 [SMILES: OCCOc1ccc(Cl)cc1] | 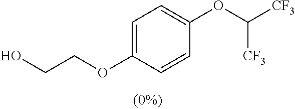 [SMILES: OCCOc1ccc(OC(C(F)(F)F)C(F)(F)F)cc1] |
| 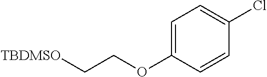 [SMILES: CC(C)(C)[Si](C)(C)OCCOc1ccc(Cl)cc1] | 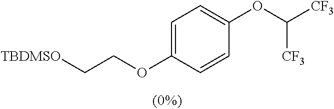 [SMILES: CC(C)(C)[Si](C)(C)OCCOc1ccc(OC(C(F)(F)F)C(F)(F)F)cc1] |
| 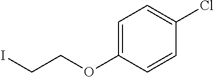 [SMILES: Clc1ccc(OCCI)cc1] | 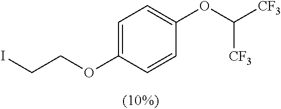 [SMILES: FC(F)(F)C(Oc1ccc(OCCI)cc1)C(F)(F)F] |
| 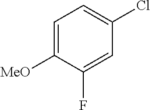 | 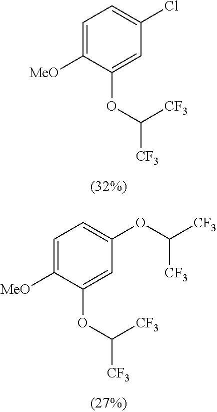 [SMILES: COc1ccc(Cl)cc1OC(C(F)(F)F)C(F)(F)F ; COc1ccc(OC(C(F)(F)F)C(F)(F)F)cc1OC(C(F)(F)F)C(F)(F)F] |
| 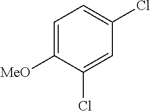 | 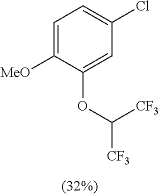 [SMILES: COc1ccc(Cl)cc1OC(C(F)(F)F)C(F)(F)F] |
| | 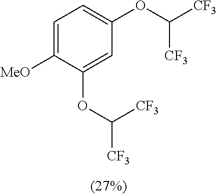 [SMILES: COc1ccc(OC(C(F)(F)F)C(F)(F)F)cc1OC(C(F)(F)F)C(F)(F)F] |
| 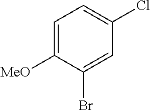 | 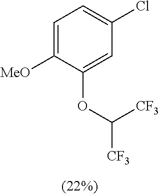 [SMILES: COc1ccc(Cl)cc1OC(C(F)(F)F)C(F)(F)F] |
| | 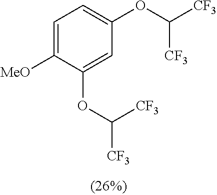 [SMILES: COc1ccc(OC(C(F)(F)F)C(F)(F)F)cc1OC(C(F)(F)F)C(F)(F)F] |
| 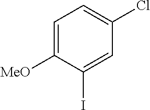 | 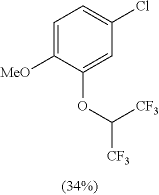 [SMILES: COc1ccc(Cl)cc1OC(C(F)(F)F)C(F)(F)F] |
| | 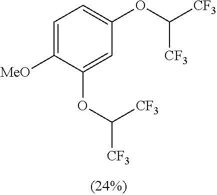 [SMILES: COc1ccc(OC(C(F)(F)F)C(F)(F)F)cc1OC(C(F)(F)F)C(F)(F)F] |
| 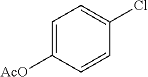 [SMILES: CC(=O)Oc1ccc(Cl)cc1] | 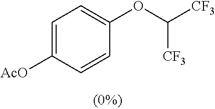 [SMILES: CC(=O)Oc1ccc(OC(C(F)(F)F)C(F)(F)F)cc1] |
| 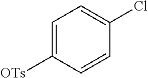 [SMILES: Cc1ccc(S(=O)(=O)Oc2ccc(Cl)cc2)cc1] | 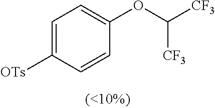 [SMILES: Cc1ccc(S(=O)(=O)Oc2ccc(OC(C(F)(F)F)C(F)(F)F)cc2)cc1] |
| 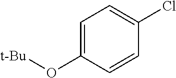 [SMILES: CC(C)(C)Oc1ccc(Cl)cc1] | 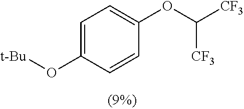 [SMILES: CC(C)(C)Oc1ccc(OC(C(F)(F)F)C(F)(F)F)cc1] |

Yields determined by 1H NMR.
* = 100% conversion

Further experiments compared para-hydroxyl, para-hydroxymethyl, para-carboxylic acid, para-formyl, para-methyl, and a meta-methyl para-hydroxyl substituted chlorobenzene. Hydroxyls capped with a methyl provided improved yield (>20%). Aryl chlorides were used as representative aryl halides.

These results show that aryl fluorinated ether formation can be achieved with mild photocatalytic conditions across varying substrates. Commercially available and bioactive griseofulvin was treated with 1,1,1,3,3,3-hexafluoropropan-2-ol, Catalyst B (1 mol %), NaHCO₃ (2 eq.), and irradiation with 440 nm light for 12 hours resulted in the desired fluorinated ether derivative of griseofulvin at 77% yield.

### Aryl Fluorinated Ether Derivative of Griseofulvin

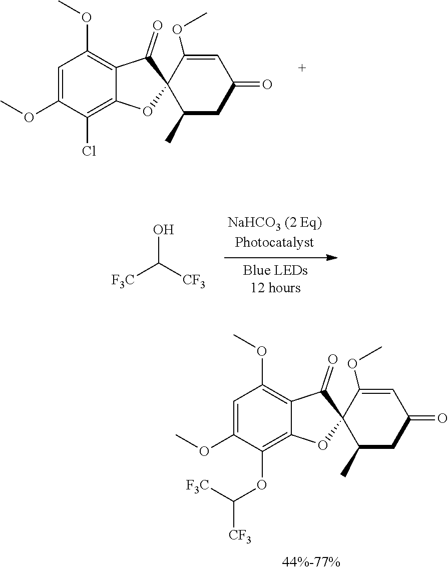

Commercially available and bioactive griseofulvin was treated with 1,1,1,3,3,3-hexafluoropropan-2-ol, photocatalyst, NaHCO₃ (2 eq.), and irradiation with 440 nm light for 12 hours resulted in the desired fluorinated ether derivative of griseofulvin. Use of 1 mol % of Catalyst B with 3 W LEDs provided an adequate 44% yield, and use of 1% mol of Catalyst B with 6 W LEDs and aluminum foil wrapped vessels provided a 77% yield. Higher catalyst loadings (5 mol % and 10 mol %) also provided comparable results, but 1 mol % ultimately provided equal or superior yields to the higher catalyst loadings. This disclosure contemplates and reasonably expects that this derivative would retain the biological activity of the original compound. This process thus provides mild conditions that are capable of furnishing fluorine containing groups on a complex bioactive molecule at suitable yields. Moreover, this approach is also suitable with other bioactives. As demonstrated in Table 5, above, a similar approach can be used to access fluorine containing derivatives of triadimefon and climbazole. Replacement of aryl halides such as griseofulvin chlorine with a fluorinated ether represents a promising path to obtaining new bioactive compounds.

### Aryl Fluorinated Ether Derivative of Clofibrate

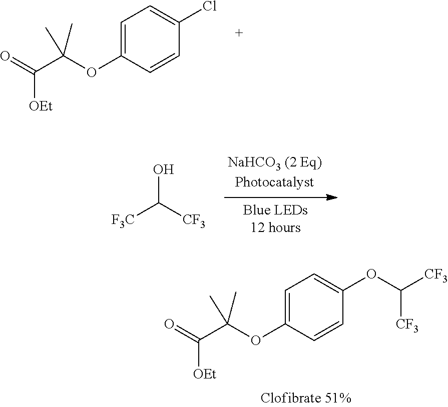 [SMILES: CCOC(=O)C(C)(C)Oc1ccc(Cl)cc1 ; OC(C(F)(F)F)C(F)(F)F ; CCOC(=O)C(C)(C)Oc1ccc(OC(C(F)(F)F)C(F)(F)F)cc1]

Commercially available and bioactive clofibrate, a cholesterol drug, was treated with 1,1,1,3,3,3-hexafluoropropan-2-ol, photocatalyst, NaHCO₃ (2 eq.), and irradiation with 440 nm light for 12 hours resulted in the desired fluorinated ether derivative of clofibrate. Use of 1% mol of Catalyst B resulted in a 51% yield. This process thus provides mild conditions that are capable of furnishing fluorine containing groups on complex bioactive drugs at suitable yields. This disclosure contemplates and reasonably expects that this derivative would retain the biological activity of the original compound.

### Aryl Fluorinated Ether Derivative of the Herbicide 2,4-D Methyl Ester

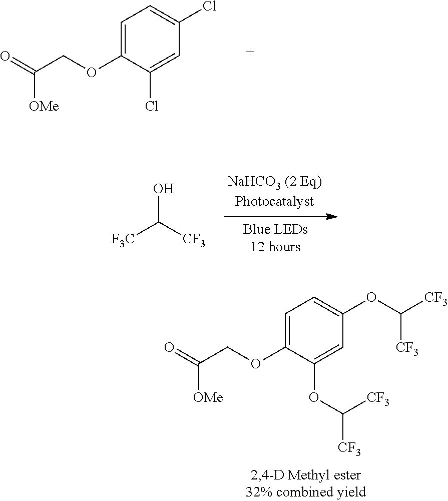 [SMILES: COC(=O)COc1ccc(Cl)cc1Cl ; OC(C(F)(F)F)C(F)(F)F ; COC(=O)COc1ccc(OC(C(F)(F)F)C(F)(F)F)cc1OC(C(F)(F)F)C(F)(F)F]

Commercially available and bioactive herbicide 2,4-D methyl ester was treated with 1,1,1,3,3,3-hexafluoropropan-2-ol, photocatalyst, NaHCO₃ (2 eq.), and irradiation with 440 nm light for 12 hours resulted in the desired fluorinated ether derivative of 2,4-D methyl ester. Use of 1% mol of Catalyst B resulted in a 32% combined yield. This process thus provides mild conditions that are capable of furnishing fluorine containing groups on complex bioactive herbicides at suitable yields, even those with multiple halogen reactive sites. This disclosure contemplates and reasonably expects that this derivative would retain the biological activity of the original compound.

### Scale Up in Flow Reactor

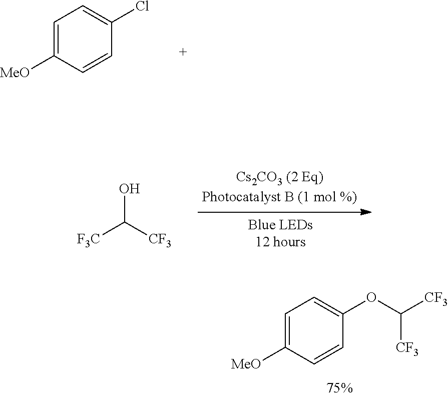 [SMILES: COc1ccc(Cl)cc1 ; OC(C(F)(F)F)C(F)(F)F ; COc1ccc(OC(C(F)(F)F)C(F)(F)F)cc1]

An example set of conditions was developed for scale up in a flow reactor at 5.4 mmol scale. Use of some bases resulted in conditions that clogged a test flow reactor. It was unexpectedly found that the use of cesium carbonate (Cs₂CO₃) was suitable for achieving high yields in a flow reactor. 4-Chloroanisole was treated with 1,1,1,3,3,3-hexafluoropropan-2-ol (0.3M), photocatalyst (1 mol % photocatalyst B), Cs₂CO₃ (2 eq.), and irradiation with 440 nm light for 12 hours under argon resulted in the desired fluorinated ether derivative, achieving 75% conversion to the desired aryl fluorinated ether.

The above examples each demonstrate an approach that eschews harsh conditions, dangerous reagents, expensive starting materials, and produces industrially relevant yields. The introduction of fluorine derivatives is significant because they are known to resist oxidation and alter the acidity and lipophilicity of biologically active compounds, yielding products with desirable properties like enhanced metabolic, oxidative, and thermal stability.

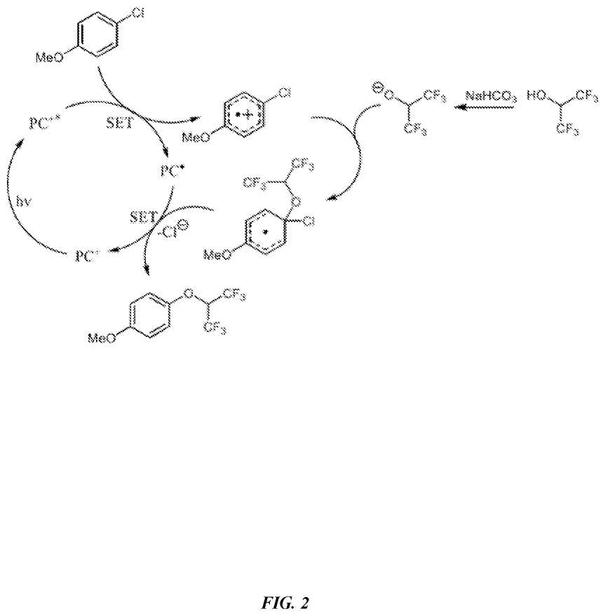

Without intending to limit to any particular mechanistic theory, FIG. 2 provides an illustration showing a proposed catalytic cycle for the observed transformations.

The mild reaction conditions of the present methods represent an important advantage. For example, the method can involve low temperatures, the use of light as a chemical reagent, a mild sodium bicarbonate base (i.e., baking soda), and can utilize aryl halide starting materials, which are both inexpensive and widely available. As a platform technology, this invention enables the development of novel drugs, agrochemicals, antibacterial, or even potential cleaning products.

### ADDITIONAL EMBODIMENTS

The following exemplary embodiments are provided, the numbering of which is not to be construed as designating levels of importance:

Embodiment 1. provides a method for preparing a fluorinated compound, comprising contacting an aryl halide with a fluorinated alcohol in the presence of a photocatalyst, a base, and irradiation with electromagnetic radiation comprising a wavelength between about 200 nm and about 800 nm.

Embodiment 2. provides the method of Embodiment 1, wherein the fluorinated alcohol is a fluorinated C1-C8 alcohol.

Embodiment 3. provides the method of any one of Embodiments 1 to 2, wherein the fluorinated alcohol comprises 1-15 fluorines.

Embodiment 4. provides the method of any one of Embodiments 1 to 3, wherein the fluorinated alcohol is a secondary alcohol.

Embodiment 5. provides the method of any one of Embodiments 1 to 4, wherein the fluorinated alcohol comprises one CH bond.

Embodiment 6. provides the method of any one of Embodiments 1 to 5, wherein the fluorinated alcohol has the structure:

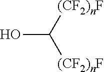

wherein n is 1 to 3.

Embodiment 7. provides the method of any one of Embodiments 1 to 6, wherein the fluorinated alcohol is hexafluoroisopropanol.

Embodiment 8. provides the method of any one of Embodiments 1 to 7, wherein the aryl halide is an aryl chloride, aryl fluoride, or an aryl bromide.

Embodiment 9. provides the method of any one of Embodiments 1 to 8, wherein the aryl halide comprises at least one of hydroxy or alkoxy.

Embodiment 10. provides the method of any one of Embodiments 1 to 9, wherein the aryl halide comprises at least one electron-donating substituent selected from the group consisting of amino, —NHalkyl, N(alkyl)₂, hydroxyl, alkoxy, acylamido, acyloxy, alkylthio, thiol, alkyl, and aryl.

Embodiment 11. provides the method of any one of Embodiments 1 to 10, wherein the aryl halide comprises methyl.

Embodiment 12. provides the method of any one of Embodiments 1 to 11, wherein the aryl halide is an agrochemical.

Embodiment 13. provides the method of any one of Embodiments 1 to 12, wherein the photocatalyst is metal-free and comprises an aromatic cation in conjugation with one or more aromatic rings.

Embodiment 14. provides the method of any one of Embodiments 1 to 13, wherein the photocatalyst is a pyrylium salt.

Embodiment 15. provides the method of any one of Embodiments 1 to 14, wherein the photocatalyst has the structure:

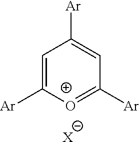

wherein Ar is an aryl group and X is an anionic counterion.

Embodiment 16. provides the method of any one of Embodiments 1 to 15, wherein the photocatalyst has the structure:

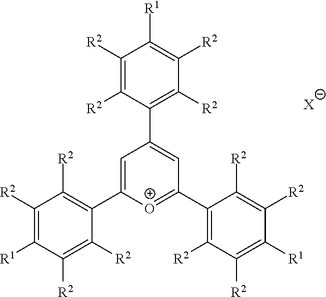

wherein
R₁ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl;
R₂ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl; and
X is an anionic counterion.

Embodiment 17. provides the method of any one of Embodiments 1 to 16, wherein the photocatalyst has the structure:

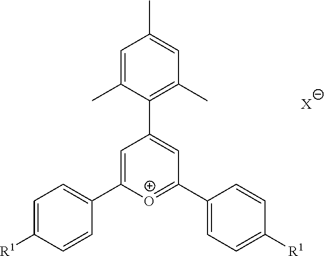

wherein R₁ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl, and X is an anionic counterion.

Embodiment 18. provides the method of any one of Embodiments 1 to 17, wherein the photocatalyst is an acridinium salt.

Embodiment 19. provides the method of any one of Embodiments 1 to 18, wherein the photocatalyst has the structure:

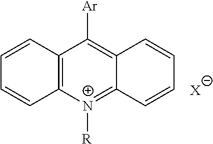

wherein Ar is an aryl group, R is alkyl or aryl, and X is an anionic counterion.

Embodiment 20. provides the method of any one of Embodiments 1 to 19, wherein the photocatalyst has the structure:

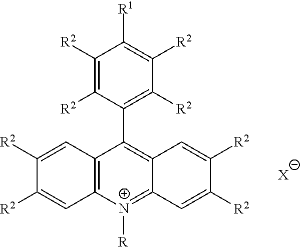

wherein R₁ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl, R₂ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl; and R is alkyl or aryl, and X is an anionic counterion, or
the photocatalyst has the structure:

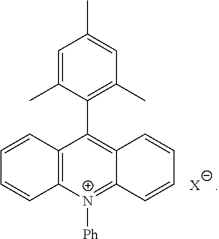

Embodiment 21. provides the method of any one of Embodiments 1 to 20, wherein the photocatalyst is used in a sub-stoichiometric amount.

Embodiment 22. provides the method of any one of Embodiments 1 to 21, wherein the photocatalyst is used in an amount less than 10 mol % relative to the aryl halide.

Embodiment 23. provides the method of any one of Embodiments 1 to 22, wherein the photocatalyst is used in an amount of about 1 mol % or less.

Embodiment 24. provides the method of any one of Embodiments 1 to 23, wherein the base is an inorganic carbonate or bicarbonate.

Embodiment 25. provides the method of any one of Embodiments 1 to 24, wherein the base has a pH of approximately 7 to approximately 10.5.

Embodiment 26. provides the method of any one of Embodiments 1 to 25, wherein the base is sodium bicarbonate.

Embodiment 27. provides the method of any one of Embodiments 1 to 26, wherein the irradiation is provided by an LED.

Embodiment 28. provides the method of any one of Embodiments 1 to 27, wherein the irradiation is provided by a blue LED.

Embodiment 29. provides the method of any one of Embodiments 1 to 28, wherein the wavelength is between about 300 nm to about 700 nm.

Embodiment 30. provides the method of any one of Embodiments 1 to 29, wherein the wavelength is about 400 nm to about 500 nm.

Embodiment 31. provides the method of any one of Embodiments 1 to 30, wherein the wavelength is about 440 nm.

Embodiment 32. provides the method of any one of Embodiments 1 to 31, wherein the base has a pH of less than 9.

Embodiment 33. provides the method of any one of Embodiments 1 to 32, wherein the contacting step is conducted at a temperature of about 40° C. or less.

Embodiment 34. provides the method of any one of Embodiments 1 to 33, wherein the contacting step is conducted at room temperature.

Embodiment 35. provides the method of any one of Embodiments 1 to 34, wherein the contacting step and irradiation step are conducted simultaneously.

Embodiment 36. provides the method of any one of Embodiments 1 to 35, wherein the contacting step is conducted in the absence of any transition metal.

Embodiment 37. provides the method of any one of Embodiments 1 to 36, wherein the contacting step is conducted in the absence of any additional catalyst or fluorination reagent.

Embodiment 38. provides a method of late-stage photochemical modification of a biologically active compound, comprising:
selecting a biologically active compound having an aryl halide; and
contacting the biologically active compound with a fluorinated alcohol in the presence of a photocatalyst, a base, and irradiation with electromagnetic radiation comprising a wavelength between about 200 nm and about 800 nm.

Embodiment 39. provides the method of Embodiment 38, wherein the biologically active compound is a pharmaceutical, an agrochemical, or a cleaning product.

Embodiment 40. provides the method of any one of Embodiments 38 to 39, wherein the agrochemical is griseofulvin.

Embodiment 41. provides a fluorinated derivative of griseofulvin having the structure:

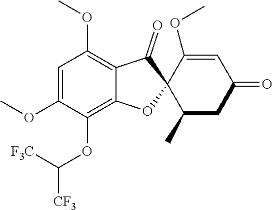

Embodiment 42. provides the method of Embodiment 40, wherein the pharmaceutical is clofibrate.

Embodiment 43. provides a fluorinated derivative of clofibrate having the structure:

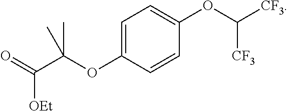 [SMILES: CCOC(=O)C(C)(C)Oc1ccc(OC(C(F)(F)F)C(F)(F)F)cc1]

Embodiment 44. provides the method of Embodiment 40, wherein the agrochemical is 2,4-D methyl ester.

Embodiment 45. provides a fluorinated derivative of 2,4-D methyl ester having the structure:

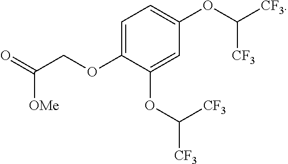 [SMILES: COC(=O)COc1ccc(OC(C(F)(F)F)C(F)(F)F)cc1OC(C(F)(F)F)C(F)(F)F]

Embodiment 46. provides the catalyst or method of any one or any combination of Embodiments 1-45 optionally configured such that all elements or options recited are available to use or select from.

### REFERENCES

1. Ogawa, Y.; Tokunaga, E.; Kobayashi, O.; Hirai, K.; Shibata, N. Current Contributions of Organofluorine Compounds to the Agrochemical Industry. iScience, 2020, 23, 101467.
2. Jeschke, P; Witschel, M.; Kramer, W.; Schirmer, U. Modern Crop Protection Compounds. John Wiley and Sons, 2019.
3. State of Wisconsin, Department of Agriculture, Trade and Consumer Protection. Wisconsin Agricultural Statistics. 2021.
4. Deller, S. Agriculture's Contribution to The Wisconsin Economy 2017, 2019.
5. Jeschke, P. The Unique Role of Fluorine in the Design of Active Ingredients for Modern Crop Protection. ChemBioChem, 2004, 5, 570-589.
6. Szabo, K.; Selander, N. Oranofluorine Chemistry: Synthesis, Modeling, and Applications. Wiley-VCH, 2021, 363-395.
7. Huang, R.; Huang, Y.; Lin, X.; Rong, M.; Weng, Z. Well-Defined Copper(I) Fluoroalkoxide Complexes for Trifluoroethoxylation of Aryl and Heteroaryl Bromides. Angewandte Chemie, 2015, 5736-5739.
8. Leroux, F.; Manteau, B.; Vors, J-P.; Pazenok, S. Trifluoromethyl ethers—synthesis and properties of an unusual substituent. Beilstein Journal of Organic Chemistry, 2008, 4, 13.
9. Donate, P.; Frederico, D. Synthesis of New Agrochemicals. Springer, 2019.

What is claimed is:

1. A method for preparing a fluorinated compound, comprising contacting an aryl halide with a fluorinated alcohol in the presence of a photocatalyst, a base, and irradiation with electromagnetic radiation comprising a wavelength between about 200 nm and about 800 nm.

2. The method of claim 1, wherein the fluorinated alcohol is a fluorinated C1-C8 alcohol.

3. The method of claim 1, wherein the fluorinated alcohol comprises 1-15 fluorines.

4. The method of claim 1, wherein the fluorinated alcohol is a secondary alcohol.

5. The method of claim 1, wherein the fluorinated alcohol comprises one CH bond.

6. The method of claim 1, wherein the fluorinated alcohol has the structure: 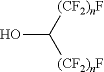
wherein n is 1 to 3.

7. The method of claim 1, wherein the fluorinated alcohol is hexafluoroisopropanol.

8. The method of claim 1, wherein the aryl halide is an aryl chloride, aryl fluoride, or an aryl bromide.

9. The method of claim 1, wherein the aryl halide comprises at least one of hydroxy or alkoxy.

10. The method of claim 1, wherein the aryl halide comprises at least one electron-donating substituent selected from the group consisting of amino, —NHalkyl, N(alkyl)₂, hydroxyl, alkoxy, acylamido, acyloxy, alkylthio, thiol, alkyl, and aryl.

11. The method of claim 1, wherein the aryl halide is an agrochemical.

12. The method of claim 1, wherein the photocatalyst is metal-free and comprises an aromatic cation in conjugation with one or more aromatic rings.

13. The method of claim 1, wherein the photocatalyst is a pyrylium salt.

14. The method of claim 1, wherein the photocatalyst has the structure: 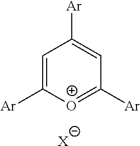
wherein Ar is an aryl group and X is an anionic counterion.

15. The method of claim 1, wherein the photocatalyst has the structure: 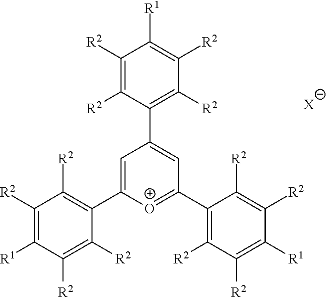
wherein
R₁ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl;
R₂ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl; and
X is an anionic counterion.

16. The method of claim 1, wherein the photocatalyst has the structure: 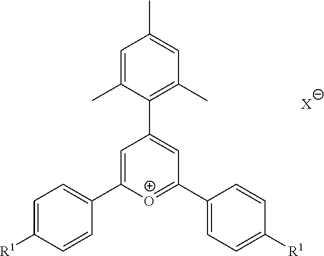
wherein R₁ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl, and X is an anionic counterion.

17. The method of claim 1, wherein the photocatalyst is an acridinium salt.

18. The method of claim 1, wherein the photocatalyst has the structure: 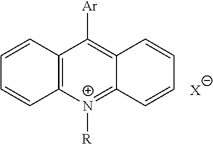
wherein Ar is an aryl group, R is alkyl or aryl, and X is an anionic counterion.

19. The method of claim 1, wherein the photocatalyst has the structure: 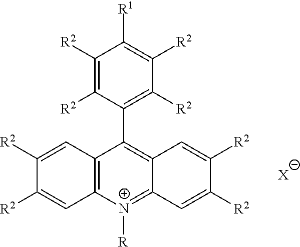
wherein
R₁ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl,
R₂ is each independently H, alkyl, aryl, halide, amino, —NHalkyl, N(alkyl)₂, hydroxyl, alkoxy, acylamido, acyloxy, alkylthio, thiol, nitro, arylsulfonyl, alkylsulfonyl, or aminosulfonyl; and
R is alkyl or aryl, and X is an anionic counterion.

20. The method of claim 1, wherein the photocatalyst has the structure: 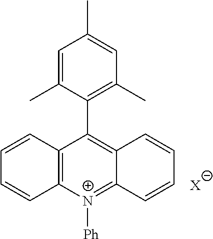
wherein X is an anionic counterion.
